(19) United States
(12) Patent Application Publication
(10) Pub. No.: US 2009/0111997 A1
(43) Pub. Date: Apr. 30, 2009

(54) Method of Generating Amorphous Solid for Water-Insoluble Pharmaceuticals

(76) Inventors: Aaron Cote, West Windsor, NJ (US); Hsien-Hsin Tung, Edison, NJ (US)

Correspondence Address: MERCK AND CO., INC, P O BOX 2000, RAHWAY, NJ, 07065-0907, US

(21) Appl. No.: 12/085,254

(22) PCT Filed: Nov. 17, 2006
(86) PCT No.: PCT/US06/44685

§ 371 Date: May 20, 2008

Related U.S. Application Data
(60) Provisional application No. 60/739,369, filed on Nov. 23, 2005.

(51) Int. Cl. C07D 235/02

(52) U.S. Cl. 548/301.7

(57) ABSTRACT

The invention encompasses a method for making an amorphous solid of a water-insoluble pharmaceutical comprising: (1) dissolving the water-insoluble pharmaceutical in a water-miscible solvent, optionally with water, to make a solution; (2)(i) rapidly mixing the solution with an antisolvent, wherein the antisolvent is water, at low temperature to precipitate an amorphous solid of the water-insoluble pharmaceutical, or (ii) rapidly mixing the solution with an antisolvent, wherein the antisolvent is water, to precipitate an amorphous solid of the water-insoluble pharmaceutical and subsequently cooling to low temperature; and (3) isolating the amorphous solid of the water-insoluble pharmaceutical. In an embodiment of the invention, the rapid mixing is conducted using an impinging jet device.

### BACKGROUND OF THE INVENTION

Water insoluble drugs, also called lipophilic, hydrophobic, etc, constitute a growing segment of the discovery and development portfolio of pharmaceutical industries. To increase the solubility of those drugs in water, one approach is to generate amorphous solid of drugs. Generally, great care must be taken to avoid drug crystallization during the preparation and the storage because amorphous solid is typically less stable in comparison to the crystalline solid.

Ways to generated amorphous solids include mechanical, thermal and solvent processes (Yu, 2001). Mechanical and thermal processes include milling/grinding (Crowley, 2001) and hot melt extrusion (Breitenbach, 2002). No solvents are involved in these processes. For solvent based methods, the drug (with or without additives) is dissolved in a solvent or solvent-water mixture. The amorphous solid is formed by rapidly removing the solvent via evaporation such as spray-drying (Broadhead, 1992), or by frozen into a total solid mass followed by vacuum drying to remove the solvent such as lypholization (Connolly, 1996) or by precipitation with an anti-solvent (Giulietti, 2001).

Each method has its limitation and advantage. For example, spray drying method is widely applicable for many drugs and different solvents. However, it is unfavorable for organic solvents with high boiling points, for example dimethyl sulfoxide which has a boiling point of 189° C. Spray drying is also not suitable for solvents which can form explosive peroxides upon drying, for example tetrahydrofuran. Precipitation is very cost effective in general and has been widely applied for the formation of amorphous inorganic salts. However, its key constraint in pharmaceutical application is maintaining the stability of amorphous solids during preparation and storage.

The current invention is a precipitation method for the generation of amorphous solid of drugs at low temperature. The stability of amorphous solid during preparation is significantly enhanced by maintaining low temperature.

### SUMMARY OF THE INVENTION

The invention encompasses a method for making an amorphous solid of a water-insoluble pharmaceutical comprising: (1) dissolving the water-insoluble pharmaceutical in a water-miscible solvent, optionally with water, to make a solution; (2)(i) rapidly mixing the solution with an antisolvent, wherein the antisolvent is water, at low temperature to precipitate an amorphous solid of the water-insoluble pharmaceutical, or (ii) rapidly mixing the solution with an antisolvent, wherein the antisolvent is water, to precipitate an amorphous solid of the water-insoluble pharmaceutical and subsequently cooling to low temperature; and (3) isolating the amorphous solid of the water-insoluble pharmaceutical. In an embodiment of the invention, the rapid mixing is conducted using an impinging jet device.

### BRIEF DESCRIPTION OF THE DRAWINGS

FIG. 1—X-ray spectra of the amorphous material, together with crystalline form I and II of the compound of Formula I. Powder X-ray diffraction is commonly used to elucidate the fraction of drug in the crystalline and amorphous form.

FIG. 2—Light microscope image of the amorphous material of the compound of Formula I. Light microscope with polarized light can show the crystallinity quickly with birefringency.

### DETAILED DESCRIPTION OF THE INVENTION

The invention is directed to a method for making an amorphous solid of a water-insoluble pharmaceutical comprising: (1) dissolving the water-insoluble pharmaceutical in a water-miscible solvent, optionally with water, to make a solution; (2)(i) rapidly mixing the solution with an antisolvent, wherein the antisolvent is water, at low temperature to precipitate an amorphous solid of the water-insoluble pharmaceutical, or (ii) rapidly mixing the solution with an antisolvent, wherein the antisolvent is water, to precipitate an amorphous solid of the water-insoluble pharmaceutical and subsequently cooling to low temperature; and (3) isolating the amorphous solid of the water-insoluble pharmaceutical.

In an embodiment of the invention, the water-insoluble pharmaceutical is a compound of Formula I

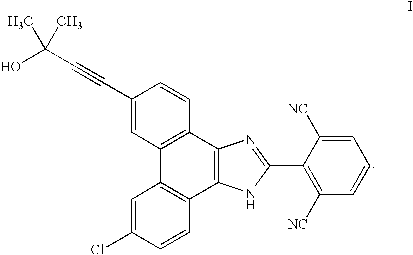

In another embodiment of the invention, the solution is rapidly mixed using an impinging jet device, mixing-T, vortex mixer, or a high speed rotor-stator homogenizer. In an aspect of the invention within this embodiment, the solution is rapidly mixed using an impinging jet device.

In another embodiment of the invention, the low temperature is within 15 degrees above the freezing temperature of the water-miscible solvent and anti-solvent mixture.

In another embodiment of the invention, the water-miscible solvent is selected from the group consisting of methanol, ethanol, acetone, acetonitrile, acetic acid, 1,4-dioxane, tetrahydrofuran (THF), diethoxymethane (DEM), dimethylsulphoxide (DMSO), N-methyl-pyrrolidone (NMP), dimethylformamide (DMF), dimethylacetamide (DMA), glycerol, ethylene glycol, and polyethylene glycol.

In another embodiment of the invention, the water-miscible solvent is a high boiling point water miscible solvent. In an aspect of the invention within this embodiment, the high boiling point water miscible solvent is selected from the group consisting of: acetic acid, 1,4-dioxane, dimethyl sulfoxide (DMSO), N-methylpyrrolidinone (NMP), dimethylformamide (DMF), dimethylacetamide (DMA), glycerol, ethylene glycol and polyethylene glycol. In another aspect of the invention within this embodiment, the high boiling point water miscible solvent is dimethyl sulfoxide

In another embodiment of the invention, the water-miscible solvent is an explosive water miscible solvent. In an aspect of the invention within this embodiment, the explosive water miscible solvent is selected from the group consisting of: tetrahydrofuran (THF) and diethoxymethane (DEM).

In another embodiment of the invention, subsequently cooling to low temperature is done by adding the slurry resulting from the rapid mixing of the solution with the antisolvent to a reservoir of antisolvent at low-temperature. In an aspect of the invention within the embodiment, the reservoir is a jacketed crystallizer.

In another embodiment of the invention, at least one inactive pharmaceutical ingredient is added to step (1) or step (2) in order to stabilize the amorphous solid of the water-insoluble pharmaceutical or improve filtration or both.

Another embodiment of the invention encompasses the method described above wherein: the water-insoluble pharmaceutical is a compound of Formula I

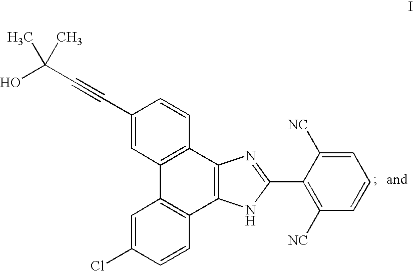

the solution is rapidly mixed using an impinging jet device.

In an aspect of the invention within this embodiment, the solution is rapidly mixed with the antisolvent, wherein the antisolvent is water, to precipitate an amorphous solid of the water-insoluble pharmaceutical and subsequently cooled to low temperature.

In another aspect of the invention within this embodiment, the solution is rapidly mixed with the antisolvent, wherein the antisolvent is water, to precipitate an amorphous solid of the water-insoluble pharmaceutical and subsequently cooled to low temperature and wherein subsequently cooling to low temperature is done by adding the slurry resulting from the rapid mixing of the solution with the antisolvent to a reservoir of antisolvent at low-temperature.

In another aspect of the invention within this embodiment, the solution is rapidly mixed with the antisolvent, wherein the antisolvent is water, to precipitate an amorphous solid of the water-insoluble pharmaceutical and subsequently cooled to low temperature and wherein subsequently cooling to low temperature is done by adding the slurry resulting from the rapid mixing of the solution with the antisolvent to a reservoir of antisolvent at low-temperature, wherein the reservoir is a jacketed crystallizer.

In another aspect of the invention within this embodiment, the water miscible solvent is dimethyl sulfoxide.

In another aspect of the invention within this embodiment, the water-miscible solvent is tetrahydrofuran.

In another embodiment of the invention, the water miscible solvent/antisolvent ratio during the rapid mixing of step (2) is in the range of 1/1 to 1/10. In an aspect of the invention within this embodiment, the water miscible solvent/antisolvent ratio is in the range of 1/2 to 1/5.

For purposes of this specification, the following terms have the indicated meanings.

The term “water insoluble pharmaceutical” means a pharmaceutical active ingredient that is insoluble or nearly insoluble in water with a dose number greater than 1. The dose number is defined as follows:

Dose number=theoretical dose in mg/water solubility×250 ml.

For example, if the theoretical dose of the drug is 20 mg per dose. For a dose number of 1, the maximum water solubility will be 25/250=0.08 mg/ml of water. Therefore, if the drug has a water solubility less than 0.08 mg/ml of water, it is considered to be water insoluble pharmaceutical. Examples of water insoluble pharmaceuticals include lovastatin (water solubility<0.01 mg/ml of water) and simvastatin (water solubility<0.01 mg/ml of water). At a hypothetic dose of 20 mg/dose, both lovastatin and simvastatin will have a dose number >8. Another example of a water insoluble pharmaceutical includes the compound of Formula I

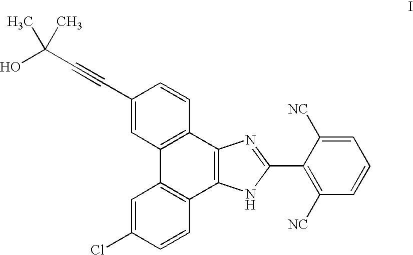

The compound of Formula I can be made as described in U.S. Provisional Application No. 60/637,180 filed Dec. 17, 2004, which is hereby incorporated by reference in its entirety, and described as follows:

### Step 1: (2E)-2-(4-bromophenyl)-3-(4-chloro-2-nitrophenyl)acrylic Acid

A 2 L flask equipped with a mechanical stirrer was charged with 183 g of 2-nitro-4-chlorobenzaldehyde, 212 g of 4-bromophenylacetic acid and 233 mL of acetic anhydride. To this solution was added 82 g of potassium carbonate and the reaction was stirred overnight at 100° C. The resulting dark mixture was cooled down to room temperature and 1.6 L of water was added followed by 800 mL of 10% HCl. The solution was decanted and taken up in water/ethyl acetate. Layers were separated, organic phase was washed with brine, dried over magnesium sulphate and volatiles were removed under reduced pressure. The residue was triturated in EtOH and the mother liquor was triturated 4 more times with EtOH to afford 219 g of the desired (2E)-2-(4-bromophenyl)-3-(4-chloro-2-nitrophenyl)acrylic acid.

### Step 2: (2E)-3-(2-amino-4-chlorophenyl)-2-(4-bromophenyl)acrylic Acid

To a 50° C. solution of 135 g of (2E)-2-(4-bromophenyl)-3-(4-chloro-2-nitrophenyl)acrylic acid from Step 1 in 1.2 L of acetic acid and 80 mL of water, was added 98 g of iron (powder) portion wise maintaining the temperature below 50° C. The mixture was stirred 2 hrs at 50° C., cooled down to room temperature, diluted with ethyl acetate (1 L) and filtered through a plug of celite. Water (1 L) was added, the layers were separated and the organic layer was washed 2 times with water, brine, dried over magnesium sulphate and volatiles were removed under reduced pressure. Residual acetic acid was removed by the addition of 1 L of H₂O to the crude mixture, the solution was filtered and washed with an additional 1 L of H₂O and finally the solid was dried under high vacuum to afford 130 g of (2E)-3-(2-amino-4-chlorophenyl)-2-(4-bromophenyl)acrylic acid.

### Step 3: 3-Bromo-6-chlorophenanthrene-9,10-dione

This quinone can be obtained by following the procedure describe in Example 36, Step 1 to 3, or by the using the following procedure: to a 0° C. solution of 118 mL of concentrated sulphuric acid in 1.0 L of water was added drop wise a solution prepared as follows: 65 g of (2E)-3-(2-amino-4-chlorophenyl)-2-(4-bromophenyl)acrylic acid from Step 2 in 1 L of water followed by the addition of 11 g of NaOH, stirring for 10 minutes at 0° C., addition of NaNO₂ (15 g) and stirring of the resulting solution at 0° C. for 20 minutes. After 30 minutes, sulfamic acid (12.5 g) was added to this mixture and after the gaz evolution seized, 1.3 L of acetone was added and the solution was stirred at 0° C. for 10 minutes. This mixture was then added to a solution of ferrocene (6.9 g) in 480 mL of acetone resulting in the formation of a green precipitate. After stirring for 20 minutes, water (2.0 L) was added, the solid was filtered and the 6-bromo-3-chlorophenanthrene-9-carboxylic acid was obtained and allowed to air dry. This crude phenanthrene was placed in 2.0 L of acetic acid followed by the addition of 54 g of CrO₃. The reaction was placed at 110° C. and after stirring for 1 hr, 18 g of CrO₃ were added. The reaction was monitored by TLC and 18 g of CrO₃ were added every hour for 3 hours where 100% conversion was observed by ¹H NMR. The mixture was cooled to room temperature, diluted in water (2.0 L), filtered and washed with water (1.0 L) to afford, after drying, 37 g of 3-Bromo-6-chlorophenanthrene-9,10-dione as a yellow solid.

### Step 4: 9-bromo-6-chloro-2-(2,6-dibromophenyl)-1H-phenanthro[9,10-d]imidazole

This imidazole was obtained following the procedure describe for Example 36, Step 4.

### Step 5: 2-(9-bromo-6-chloro-1H-phenanthro[9,10-d]imidazol-2-yl)isophthalonitrile

This imidazole was obtained following the procedure describe for Example 36, Step 5.

### Step 6: 2-[9-chloro-6-(3-hydroxy-3-methylbut-1-yn-1-yl)-1H-phenanthro[9,10-d]imidazol-2-yl]isophthalonitrile

To a solution of 13 g of 2-(9-bromo-6-chloro-1H-phenanthro[9,10-d]imidazol-2-yl)isophthalonitrile in 240 mL of DMF is added 5.5 mL of 2-methyl-3-butyn-2-ol, 2.0 g of tetrakis(triphenylphosphine)palladium, 1.1 g of copper iodide and 5.6 mL of diisopropylamine. The mixture is stirred at 55° C. for 1 hr then cooled to room temperature and diluted with ethyl acetate (250 mL). Water (250 mL) is added and the layers were separated, the organic phase is washed with brine, dried over magnesium sulphate and volatiles are removed under reduced pressure. The crude mixture is then purified on silica gel using 50% hexane/ethyl acetate. The product is then recrystallized in THF and triturated in hot ethyl acetate/ether mixture to afford 5.4 g of [9-chloro-6-(3-hydroxy-3-methylbut-1-yn-1-yl)-1H-phenanthro[9,10-d]imidazol-2-yl]isophthalonitrile as a light yellow solid. ¹H NMR (Acetone-d₆): 8.93 (s, 2H), 8.53 (m, 2H), 8.36 (d, 2H), 8.01 (t, 1H), 7.78 (d, 2H), 4.53 (s, 1H), 1.61 (s, 6H).

For reference, Example 36 is as follows:

### EXAMPLE 36

### 2-(6-bromo-9-chloro-1H-phenanthro[9,10-d]imidazol-2-yl)isophthalonitrile

### Step 1: 1-bromo-4-[2-(4-chlorophenyl)vinyl]benzene

To a solution of (4-bromobenzyl)triphenylphosphonium bromide (396 g; 0.77 mol) in 2.5 L of DMF at 0° C., was added 37 g (0.92 mol) of NaH (60% in oil) in four portions. The solution was stirred 1 hr at 0° C. followed by the addition of 109 g (0.77 mol) of 4-chlorobenzaldehyde in two portions. This mixture was warmed up to room temperature, stirred 1 hr and quench by pouring the reaction into a 5° C. mixture of 10 L of water and 2.5 L of Et₂O. Aqueous layer was extracted with Et₂O, combined organic layers were washed with brine and dried over Na₂SO₄. Volatiles were removed under reduced pressure and the residue was dissolved in 1.5 L of cyclohexane and filtered through a pad of silica gel (wash with cyclohexane). 16 g of one isomer crystallized out of the solution as a white solid and after evaporation of the volatiles, 166 g of the other isomer 1-bromo-4-[2-(4-chlorophenyl)vinyl]benzene was isolated.

### Step 2: 3-bromo-6-chlorophenanthrene

A 2 L vessel equipped with a pyrex inner water-cooled jacket was charged with 5.16 g (17 mmol) of 1-bromo-4-[2-(4-chlorophenyl)vinyl]benzene from Step 1, 2 L of cyclohexane, 25 μmL of THF, 25 mL of propylene oxide and 6.7 g (26 mmol) of iodine. The stirring solution was degassed by bubbling nitrogen and was exposed to UV light for 24 hrs by inserting a 450 W medium pressure mercury lamp in the inner. The reaction was quenched with 10% Na₂S₂O₃ and aqueous layer was extracted with ethyl acetate. Combined organic layers were washed with brine, dried over Na₂SO₄ and volatiles were removed under reduced pressure. The residue was swished in a minimal amount of ethyl acetate to afford approx. 5 g of 3-bromo-6-chlorophenanthrene as a solid.

### Step 3: 3-Bromo-6-chlorophenanthrene-9,10-dione

To a solution of 3-bromo-6-chlorophenanthrene from Step 2 (1.71 g; 5.86 mmol) in 35 mL of acetic acid was added 2.3 g (23.5 mmol) of CrO₃. The mixture was stirred 2 hrs at 100° C., cooled down to room temperature, poured into 300 mL of water and stirred for 1 hr. The suspension was filtered, washed with water and Et₂O and pumped under reduced pressure to afford 1.67 g of 3-bromo-6-chlorophenanthrene-9,10-dione as a solid.

### Step 4: 9-bromo-6-chloro-2-(2,6-dibromophenyl)-1H-phenanthro[9,10-d]imidazole

To a solution of 15.5 g of 3-bromo-6-chlorophenanthrene-9,10-dione from Step 3 in 400 mL of acetic acid, was added 74.2 g of ammonium acetate and 19.1 g of 2,6-dibromobenzaldehyde. The mixture was stirred overnight at 120° C., cooled down to room temperature diluted in 4 L of water and filtered. The resulting solid was refluxed 2 hrs in toluene with a Dean Stark apparatus. After cooling down to room temperature, the suspension was filtered, the solid washed with toluene and the resulting beige solid dried under high vacuum to produce 26 g of 9-bromo-6-chloro-2-(2,6-dibromophenyl)-1H-phenanthro[9,10-d]imidazole.

### Step 5: 2-(9-bromo-6-chloro-1H-phenanthro[9,10-d]imidazol-2-yl)isophthalonitrile

To a solution of 26 g of 9-bromo-6-chloro-2-(2,6-dibromophenyl)-1H-phenanthro[9,10-d]imidazole from Step 4 in 200 mL of dry DMF, was added 14.2 g of CuCN. The reaction was stirred overnight at 85° C., cooled down to room temperature, brine was added and the mixture stirred for 30 minutes. The solution was diluted in ethyl acetate, washed with 10% ammonium hydroxide, brine, dried over sodium sulphate and volatiles were removed under reduced pressure to afford 26 g of 2-(9-bromo-6-chloro-1H-phenanthro[9,10-d]imidazol-2-yl)isophthalonitrile as a solid. ¹H NMR (Acetone-d₆): 9.19 (s, 1H), 9.02 (s, 1H), 9.71 (bs, 1H), 8.49 (bs, 1H), 8.39 (d, 2H), 8.07 (t, 1H), 7.97 (d, 1H), 8.81 (d, 1H).

An alternate method for making the compound of Formula I is as follows:

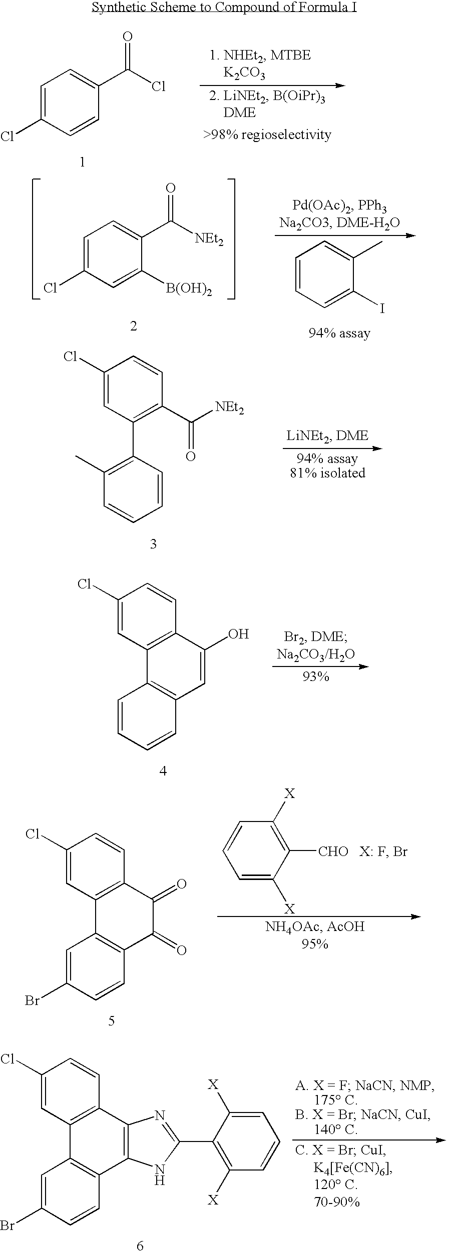

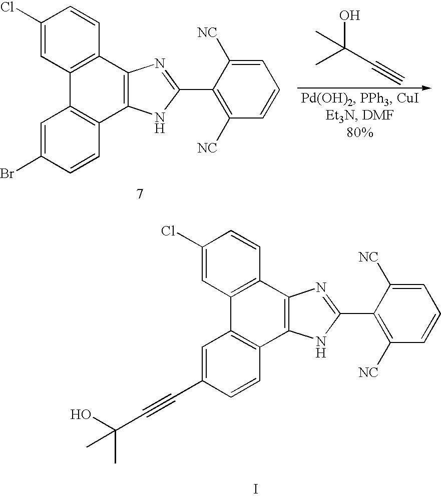

### Experimental Procedure

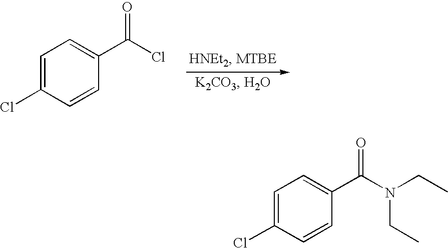

To a round bottom flask was charged potassium carbonate (65 g, 469.7 mmol), H₂O (400 mL), MTBE (800) and diethyl amine (81 mL, 861.1 mmol). p-Chlorobenzoyl chloride (100 mL, 782.8 mmol) was then added over 30 minutes, maintaining the temperature under 25° C. After addition, the phases were separated and the organics washed with brine (200 mL). The solution was then solvent switched to DME to give a crude solution of the amide, which was used directly in the next step.

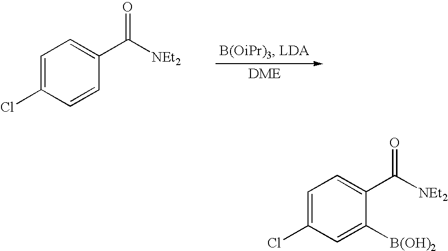

To the crude solution of the amide (10 g, 47.3 mmol) in 7.5 mL/g DME (75 mL) was added triisopropyl borate (19.5 mL, 85.1 mmol) and the resulting solution was cooled to −25° C. A freshly prepared 1.45 M solution of lithium diethylamide (45.6 mL, 66.2 mmol) was then added dropwise over 30 minutes. [NOTE: Lithium diethylamide was generated by treatment of diethylamine in THF with a 2.5M solution of n-butyllithium in hexanes, maintaining the temperature below 0° C. during the addition] At the end of addition, the mixture was aged for additional 15 minutes, at which all starting material has been consumed to give the corresponding boronic acid in >98% regioselectivity. The crude solution was then used directly in the next step.

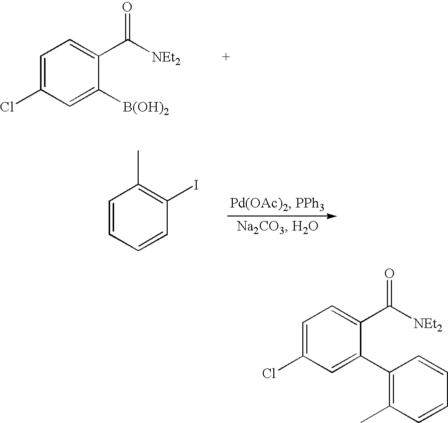

To the crude solution of boronic acid as obtained above was added degassed water (95 mL) at 0° C. and solid Na₂CO₃ (13.5 g, 127.7 mmol). To the resulting suspension was successively added PPh₃ (223 mg, 0.85 mmol), 2-iodotoluene (5.4 mL, 42.6 mmol) and Pd(OAc)₂ (95.5 mg, 0.43 mmol) and the mixture was degassed, heated to 70° C. and aged for 6 hours, at which complete consumption of 2-iodotoluene was typically observed. At the end of reaction, MTBE (75 mL) was added and the resulting slurry was filtered. Sodium chloride was added to the biphasic filtrate to ease the separation and the layers were cut. The organic phase was washed one time with water (20 mL) and brine (2×30 mL). The crude solution was then concentrated, solvent switched to DME and used directly in the next step. Typical assay yield: 90-94%.

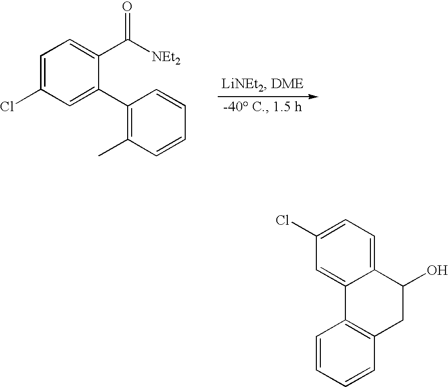

To the crude solution of the amide (13.9 g, 46.2 mmol) in 7.5 mL/g DME (104 mL), kept at −45° C., was added freshly prepared 1.44 M solution of LiNEt₂ in THF (41.7 mL, 60 mmol) over 15 min. The resulting brown solution was aged for 75 minutes, at which complete consumption of starting material was observed by HPLC. MTBE (120 mL) was added followed by slow addition of 6N HCl (30.8 mL, 184.7 mmol). The resulting mixture was allowed to warm to RT and the layers were separated (pH of the aqueous layer should be 2-3). The organic layer was washed one time with H₂O (55 mL), brine (60 mL), concentrated and solvent switched to toluene for crystallization. When approximately 4 mL/g of product in a 3:1 mixture of toluene:DME was obtained, the slurry was refluxed to dissolve all the solid, cooled slowly to 60° C. and treated with 5 mL/g of methyl cyclohexane (crystals are typically formed at 75-80° C.) over 1 hour, while allowing the mixture to cool to RT. The slurry was then concentrated to give a volume of 3.5 mL/g of product and then re-treated with 2 mL/g of methyl cyclohexane over 0.5 hour. The slurry was aged at 0° C. for 0.5 hour, filtered and the wetcake was washed with a cold 3:1 mixture of toluene:methyl cyclohexane, followed by drying under constant flow of N₂. The desired product was obtained as light tan solid in 81% yield.

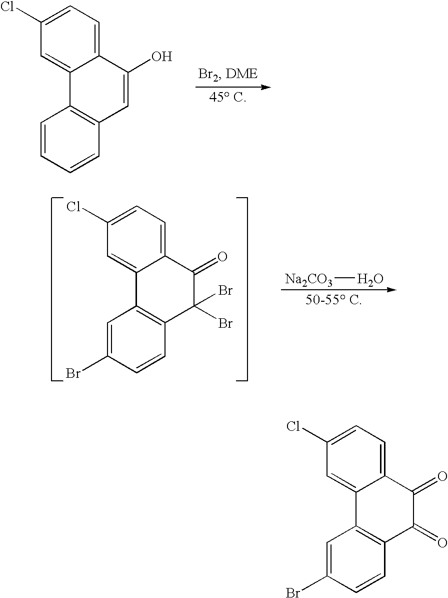

To a solution of chloro-phenanthrole (41 g, 179.8 mmol) in dry DME (600 mL, KF=25 ppm, solution KF=1000 ppm) at 15° C. was added Br₂ (32.3 mL, 629.4 mmol) over 20 minutes, at which a 15° C. exotherm was evident during the addition. The resulting suspension was then warmed to 40-45° C. and aged for 4 hours to give a clear, red solution. A solution of Na₂SO₃ (4.4 g, 36 mmol) in 30 mL of H₂O was added, followed by a solution of Na₂CO₃ (57 g, 539.4 mmol) in 250 mL H₂O. The resulting suspension was warmed to 55° C. and aged for 5 hour, at which a complete hydrolysis was obtained (additional of H₂O might be necessary to re-dissolve precipitated Na₂CO₃). The reaction mixture was then concentrated at 35-40° C. (35-40 torr) to about a third of its volume and the slurry was filtered, washed with H₂O (80-100 mL), followed by 1:1 DME:H₂O (100 mL) and dried under constant flow of N₂. The solid obtained was generally pure enough for the next step; typical yield: 93%.

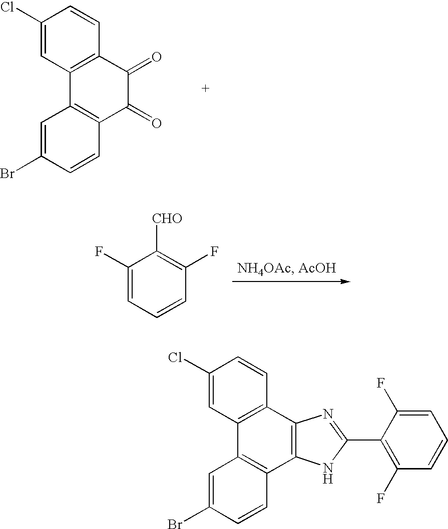

The chlorobromodiketone (4.54 g, 14.12 mmol), difluorobenzaldehyde (1.5 mL, 14.12 mmol), and ammonium acetate (21.77 g, 282.38 mmol) were charged to a 250 mL round bottom three neck flask under nitrogen. Acetic acid (90 mL) was added with stirring, and the slurry was heated to 120° C. for 1 hour. The slurry was then cooled to room temperature and water (90 mL) was added over 30 min. Upon completion of addition of water, the reaction mixture was filtered, washed with water (45 mL), and dried overnight under nitrogen and vacuum to give the acetic acid salt as a yellow solid.

In order to obtain the freebase, the crude product was dissolved in 1:1 THF/MTBE (90 mL) and charged to a 250 mL flask along with 1N NaOH (45 mL). The mixture was then heated to 40° C. for one hour. The phases were cut at 40° C., and the organic layer washed with 1N NaOH (45 mL). The organic layer was then concentrated, solvent switched to MTBE, and brought to a final volume of 45 mL. The reaction mixture was slurried at 35° C. for one hour, cooled to room temperature, filtered, washed with MTBE (23 mL), and dried under nitrogen. The difluoro imidazole freebase (5.97 g) was obtained as a light yellow solid in 95% isolated yield.

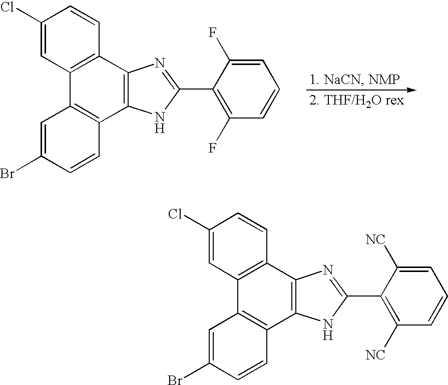

Method A: The difluoroimidazole (6.79 g, 13.39 mmol) and sodium cyanide (3.28 g, 66.95 mmol) were charged to a 500 mL round bottom flask under nitrogen. N-methylpyrrolidone (NMP, 60 mL) was added with stirring, and the slurry was heated to 175° C. for 28 hours. The reaction mixture was then cooled to room temperature. Water (240 mL) was added over 2 hours, and the slurry was allowed to stir for 48 hours. Sodium chloride (36 g) was added to the slurry and it was stirred for additional 2 hours. The slurry was then cooled to 0° C., stirred for 1 hour, filtered, and washed with water (30 mL). The wetcake was then dried under nitrogen to give the desired product as NMP solvate.

The solid was slurried in THF (42 mL, 7.5 mL/g) at 65° C. for 1 hour. The mixture was then cooled to room temperature, followed by addition of water (14 mL, 2.5 mL/g) over 1 hour. The slurry was then concentrated under vacuum, removing 14 mL of solvent and the resulting slurry was filtered. The wetcake was washed with 1:1 THF/H₂O (14 mL), and dried under nitrogen. The desired product (3.83 g) was obtained as THF solvate in 54% isolated yield.

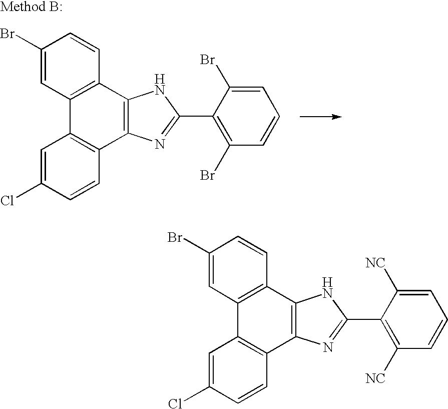

1.0 g of tribromoimidazole freebase (1.8 mmol), 260 mg NaCN (5.3 mmol), 135 mg CuI (0.71 mmol) and 7 mL DMF were combined and degassed, then heated to 120° C. for 45 h. 7 mL of 6:1 water:NH₄OH was added, and the crude product was isolated by filtration. After drying, the material was recrystallized from 1:1 THF:MTBE (16 mL) to afford 870 mg of the dicyano product as the THF solvate (97%).

Method C: tribromoimidazole AcOH salt (1.30 g, 87 wt % as free base, 2 mmol) was treated with K₄[Fe(CN)₆].3H₂O (845 mg, 2 mmol, finely-powdered), CuI (76.2 mg, 0.4 mmol), and 1,2-phenylenediamine (43.3 mg, 0.4 mmol) in DMF (5.7 mL). The reaction mixture was heated to 135° C. for 36 h, diluted with DMF (5.7 mL), and filtered when hot. The solid was washed thoroughly with acetone, and the washes were combined with the filtrate. The organic solution was concentrated to remove acetone, and H2O (2.8 mL) was added over 15 min at RT. The resulting solid was collected by filtration, washed with H2O, and to afford brown solid (1.06 g). The crude solid was then stirred in THF (4 mL) at 60° C. for 1 h and allowed to cool to RT. The resulting solid was collected by filtration, washed with hexane, and dried to afford dicanide THF solvate as off white powder (864 mg, 89.5 wt %).

For Methods B and C above, the tribromoimidazole compound is made following the procedure described above for making the difluoroimidazole compound, but substituting dibromobenzaldehyde for difluorobenzaldehyde.

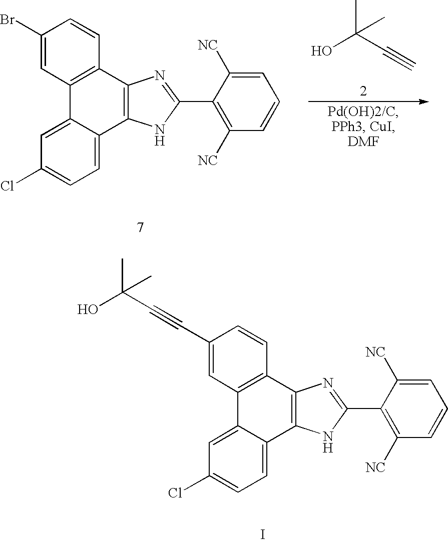

A 7 ml vial, equipped with stir bar and septum screw cap was charged with 6.2 mg of 20 wt % Pd(OH)₂ on carbon containing about 16 wt % water (about 1.0 mg Pd(OH)₂ corrected for solid support and water), 69 mg compound 7, 8 mg triphenylphosphine, and 6 mg copper(I) iodide. The vial was brought into a nitrogen filled glovebox where the remaining nitrogen-purged reaction materials were added. N,N-Dimethylformamide (0.68 mL) was charged followed by 2-methyl-3-butyn-2-ol (0.022 mL) and triethylamine (0.031 mL). The vial was sealed, removed from the glovebox, placed in a heating block equipped with a nitrogen-purged cover attached, and warmed to an external temperature of 52° C. The reaction was agitated with heating for about 17 h. HPLC analysis of the reaction at this time showed about 95% LCAP conversion to the compound of formula I using an external reference with >99 LCAP conversion of bromide 7 @ 210 nm.

The compound of Formula I is a selective inhibitor of the microsomal prostaglandin E synthase-1 enzyme and is therefore useful to treat pain and inflammation. Dosage levels range from about 0.01 mg to about 140 mg/kg of body weight per day, including dosage unit forms containing 1, 10 or 100 mg.

The term “low temperature” means a temperature in the range of below 10 degrees to above 15 degrees relative to the freezing temperature of the water-miscible solvent/anti-solvent mixture. This freezing temperature is easily discerned by one having ordinary skill in the art. For example, the freezing temperature of dimethyl sulfoxide and water mixture can be determined using the following diagram (Gaylord Chemical Corporation, Technical Bulletin, dimethyl sulfoxide). According to the diagram, the pure water has a freezing point of 0° C., and the pure dimethyl sulfoxide has a freezing point of 18° C. 20° C. (by the accuracy of the diagram below). For a solvent mixture of 20 wt % dimethyl sulfoxide in water, the freezing point would be in between −5° C. and −7° C. (by the accuracy of the diagram below).

The term “rapidly mixing” can be accomplished using a variety of devices such as a jet impinging device, a mixing-T, a vortex mixer, or a high speed rotor/stator homogenizer, etc. These devices and methods for operating these devices are well known by those having ordinary skill in the art. An impinging jet device, for example, is described in U.S. Pat. No. 5,314,506, granted May 24, 1994.

The slurry resulting from the rapid mixing of the solution with the antisolvent can be “subsequently cooled” by a variety of means well known by the ordinarily skilled artisan. Subsequent cooling can be accomplished by adding the slurry to a cold reservoir of anti-solvent at low temperature. Examples include a jacketed crystallizer, which is commercially available.

The amorphous solid of the water-insoluble pharmaceutical can be isolated by a variety of techniques, such as filtration, centrifugation, and membrane filtration, etc.

The term “water miscible solvent” means solvent which is miscible with water at a solvent composition less than 50 wt % of the solvent/water mixture. Examples of water miscible solvents include alcohols such as, methanol, ethanol; ketones such as acetone and various other solvents such as acetonitrile, acetic acid, tetrahydrofuran (THF), diethoxymethane (DEM), 1,4-dioxane, dimethylsulphoxide (DMSO), N-methyl-pyrrolidinone (NMP), dimethylformamide (DMF), and dimethylacetamide (DMA), glycerol, (poly)ethylene glycol, and the like.

An embodiment of the invention encompasses the use of a “high boiling point water miscible solvent” which means a water miscible solvent with a boiling point higher than 100° C., or use of an “explosive water miscible solvent” which means a water miscible solvent with a potential to form explosive peroxides upon drying/evaporation. Examples of “high boiling point water miscible solvents” include acetic acid, 1,4-dioxane, dimethyl sulfoxide (DMSO), N-methylpyrrolidinone (NMP), dimethylformamide (DMF), dimethylacetamide (DMA), glycerol, (poly)ethylene glycol etc. Examples of “explosive water miscible solvent” include tetrahydrofuran (THF), diethoxymethane (DEM) and various ethers etc.

The invention will now be illustrated by the following non-limiting examples:

### EXAMPLE 1

2 grams of the compound of Formula I solid and 10 ml of dimethyl sulfoxide (DMSO) solvent were charged into a glass flask at room temperature. All solids were dissolved. The solution was mixed rapidly with 20 to 30 ml of water (as anti-solvent) using an impinging jet device, similar to the one disclosed in U.S. Pat. No. 5,314,506, granted May 24, 1994, to precipitate the compound of Formula I as amorphous material. The ratio of DMSO to water ratio at the impingement ranges from 1/2 to 1/3. The resulting slurry was sent to a jacketed crystallizer which contained 30-20 ml of water under agitation. The final DMSO/water ratio is maintained at 1/5. The temperature of the batch was maintained at −5° C. to 5° C. to maintain the stability of amorphous solid of the compound of Formula I in slurry. The slurry was filtered and washed with water at 0° C.-5° C. The wet cake was vacuum dried. The crystallinity of the cake was examined by X-ray diffraction analysis and light microscope. The residual solvent in the cake was analyzed by GC.

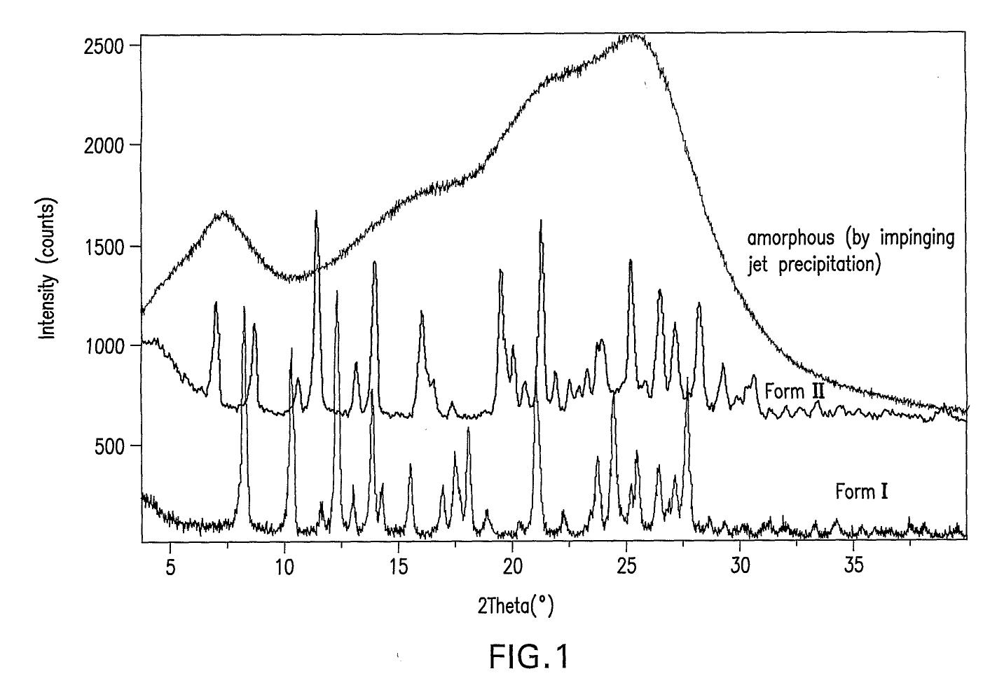

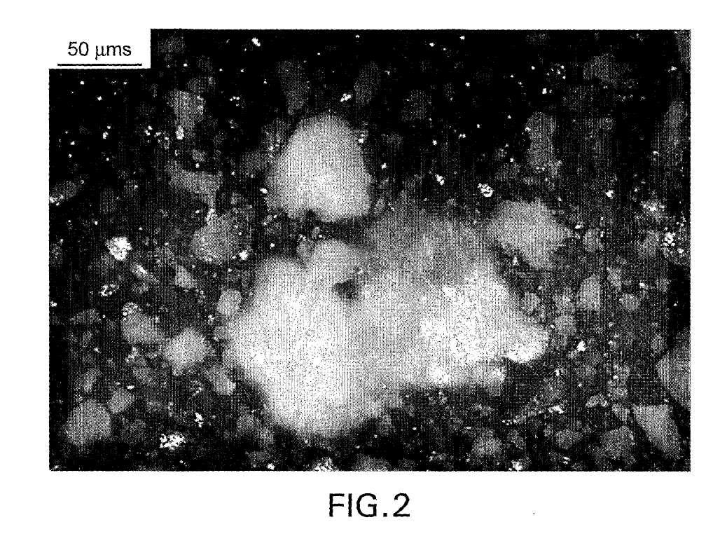

The amorphous solid of the light microscopic image (FIG. 2) are mainly non-birefringent with some birefringent crystals. GC analysis of the amorphous solid shows <0.5 wt % residual DMSO in the solid. X-ray spectra of the amorphous material, together with crystalline form I and II of the compound of Formula I are shown in FIG. 1.

### EXAMPLE 2

To a 125 mL jacketed crystallizer equipped with an IKA-Works rotor/stator homogenizer (model T25 with fine dispersion element) as the agitator, charge 50 mL DI water. Turn on the homogenizer at 9.1 m/s tip speed and adjust the jacket temperature until water temperature in vessel is 0° C. to 2° C. Dissolve 1 gram of the compound of Formula I in 5 ml THF in a separate 50 ml glass flask, then add this solution to the above 125 ml crystallizer over 5 minutes. Following charge, adjust jacket temperature of the above crystallizer to achieve 0-2° C. batch temperature. Filter batch and wash with cold water. Dried sample was analyzed by XRD which confirmed that material was amorphous.

### REFERENCES

Breitenbach, Jorg, “Melt Extrusion: From Process to Drug Delivery Technology”, European J. of Pharm. & Biopharm., 54, p 107 (2002)
Yu, Lian, “Amorphous Pharmaceutical Solids: Preparation, Characterization, and stabilization,” Adv. Drug Delivery Review, 48, p 27-42 (2001).
Broadhead, J., S. K. Rouan Edmond, C. t. Rhodes, “The Spray Drying of Pharmaceuticals,” Drug Dev. Ind. Pharm., 18, p 1169 (1992).
Connolly, Michael, P. G. Debenedetti, Hsien-Hsin Tung, “Freeze Crystallization of Imipenem”, J. of Pharm. Science, 85, p 174 (1996).
Crowley, K. J., and G. Zografi, “Cryogenic Grinding of Indomethacin Polymorphs and Solvates: Assessment of Amorphous Phase Formation and Amorphous Phase Physical Stability,” J. of Pharm. Sciences, 91, p 492 (2002)
Giulietti, M., et al., “Industrial Crystallization and Precipitation from Solutions: State of the Technique,” Braz. J. Chem. Eng. 18 (4) (2001)

What is claimed is:

1. A method for making an amorphous solid of a water-insoluble pharmaceutical comprising:
(1) dissolving the water-insoluble pharmaceutical in a water-miscible solvent, optionally with water, to make a solution;
(2) (i) rapidly mixing the solution with an antisolvent, wherein the antisolvent is water, at low temperature to precipitate an amorphous solid of the water-insoluble pharmaceutical, or
(ii) rapidly mixing the solution with an antisolvent, wherein the antisolvent is water, to precipitate an amorphous solid of the water-insoluble pharmaceutical and subsequently cooling to low temperature; and
(3) isolating the amorphous solid of the water-insoluble pharmaceutical.

2. The method according to claim 1 wherein the water-insoluble pharmaceutical is a compound of Formula I

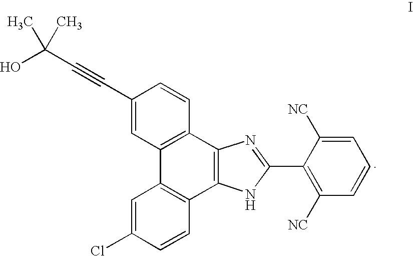

3. The method according to claim 1 wherein the solution is rapidly mixed using an impinging jet device, mixing-T, vortex mixer, or a high speed rotor-stator homogenizer.

4. The method according to claim 3 wherein the solution is rapidly mixed using an impinging jet device.

5. The method according to claim 1 wherein the low temperature is within 15 degrees above the freezing temperature of the water-miscible solvent and anti-solvent mixture.

6. The method according to claim 1 wherein the water-miscible solvent is selected from the group consisting of methanol, ethanol, acetone, acetonitrile, acetic acid, 1,4-dioxane, tetrahydrofuran (THF), diethoxymethane (DEM), dimethylsulphoxide (DMSO), N-methyl-pyrrolidone (NMP), dimethylformamide (DMF), dimethylacetamide (DMA), glycerol, ethylene glycol, and polyethylene glycol.

7. The method according to claim 1 wherein the water-miscible solvent is a high boiling point water miscible solvent.

8. The method according to claim 7 wherein the high boiling point water miscible solvent is selected from the group consisting of: acetic acid, 1,4-dioxane, dimethyl sulfoxide (DMSO), N-methylpyrrolidinone (NMP), dimethylformamide (DMF), dimethylacetamide (DMA), glycerol, ethylene glycol and polyethylene glycol.

9. The method according to claim 8 wherein the high boiling point water miscible solvent is dimethyl sulfoxide

10. The method according to claim 1 wherein the water-miscible solvent is an explosive water miscible solvent.

11. The method according to claim 10 wherein the explosive water miscible solvent is selected from the group consisting of: tetrahydrofuran (THF) and diethoxymethane (DEM).

12. The method according to claim 1 wherein subsequently cooling to low temperature is done by adding the slurry resulting from the rapid mixing of the solution with the antisolvent to a reservoir of antisolvent at low-temperature.

13. The method according to claim 12 wherein the reservoir is a jacketed crystallizer.

14. The method according to claim 1 wherein at least one inactive pharmaceutical ingredient is added to step (1) or step (2) in order to stabilize the amorphous solid of the water-insoluble pharmaceutical or improve filtration or both.

15. The method according to claim 1 wherein:
the water-insoluble pharmaceutical is a compound of Formula I 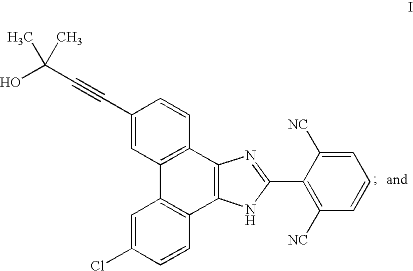
the solution is rapidly mixed using an impinging jet device.

16. The method according to claim 15 wherein the solution is rapidly mixed with the antisolvent, wherein the antisolvent is water, to precipitate an amorphous solid of the water-insoluble pharmaceutical and subsequently cooled to low temperature.

17. The method according to claim 16 wherein subsequently cooling to low temperature is done by adding the slurry resulting from the rapid mixing of the solution with the antisolvent to a reservoir of antisolvent at low-temperature.

18. The method according to claim 17 wherein the reservoir is a jacketed crystallizer.

19. The method according to claim 15 wherein the water miscible solvent is dimethyl sulfoxide.

20. The method according to claim 15 wherein the water-miscible solvent is tetrahydrofuran.

21. The method according to claim 1 wherein the water miscible solvent/antisolvent ratio during the rapid mixing of step (2) is in the range of 1/1 to 1/10.

22. The method according to claim 21 wherein the water miscible solvent/antisolvent ratio is in the range of 1/2 to 1/5.
